(19) United States
(12) Patent Application Publication
(10) Pub. No.: US 2007/0190650 A1
(43) Pub. Date: Aug. 16, 2007

(54) COMPOSITE BODY OF INORGANIC COMPOUND AND CELLS AND PRODUCTION METHOD THEREOF

(75) Inventors: Akira YAMAMOTO, Tokyo (JP); Yusuke IIMORI, Tokyo (JP); Mae KOYAMA, Tokyo (JP)

Correspondence Address: GREENBLUM & BERNSTEIN, P.L.C., 1950 ROLAND CLARKE PLACE, RESTON, VA, 20191, US

(73) Assignee: PENTAX CORPORATION, Tokyo (JP)

(21) Appl. No.: 11/673,793

(22) Filed: Feb. 12, 2007

(30) Foreign Application Priority Data
Feb. 15, 2006 (JP) 2006-037554

(51) Int. Cl. C12N 5/06

(52) U.S. Cl. 435/325

(57) ABSTRACT

A composite body of an inorganic compound and cells includes animal cells filled with fine particles of the inorganic compound, and the fine particles of the inorganic compound adhered to an outer portion of the animal cells. A method of producing a composite body of an inorganic compound and cells is also provided, including cultivating animal cells in a culture medium containing fine particles of the inorganic compound, and separating a solid component from the cultivated product.

### BACKGROUND OF THE INVENTION

1. Field of the Invention

The present invention relates to a composite body having an inorganic compound and cells as well as a method of producing the same.

2. Description of the Related Art

In the field of orthopedic surgery, the mainly adopted method for compensating a missing portion of a bone includes transplantation of an autologous bone or a bone from another person, and the implantation of an artificial bone formed from ceramics, metal, resin or a composite thereof, alone or in combination with an autologous bone. The transplantation of an autologous bone involves the transplantation of a living body bone obtained from the patient's living body, and thus the transplanted bone can be most safely and rapidly bonded to the bone of the missing portion. Among the available transplantation methods, the most frequently adopted method is the method of transplanting a part of the iliac bone. However, there are limitations in the size and configuration of the part of the iliac bone which can be obtained, and also there is a problem with the patient being adversely affected by the excision of his or her healthy bone portion. Accordingly, transplantation of an autologous bone is recognized as being troublesome with regard to the above points.

New materials for compensating for or implanting into missing bones are being researched. In recent years, such materials include a composite body having a composition similar to that of bone, which includes collagen and synthesized calcium phosphate, and an artificial material having impregnated therein a physiological active substance for accelerating bone formation. Furthermore, implanting bone material composed of a scaffold material with previously seeded bone marrow cells has also been researched. Since such implanting bone materials are embedded in the living body, the development of these implanting bone materials is carried out with the intension of providing a function of rapidly and strongly bonding the embedded bone material to the living bone around the missing portion, in addition to having nontoxic properties and being provided safely in the living body.

Furthermore, the target functions of the new implanting bone materials which are considered to be important include a capability of inducing an autologous bone, bone formation capability, and mechanical strength of the bone materials at the initial fixing stage thereof. To provide an artificial bone having these functions which is similar to a living bone, for example, utilization of the stem cell capable of being differentiated into osteoblast cells, has been tried.

When stem cells of the patient is used in the implantation process, the method of collecting cells from the bone marrow of the patient is often employed. More specially, the methods developed for attaining this method include the method of collecting cells from the bone marrow and then adhering the cells to, and culturing the cells in, the scaffold material under in vitro conditions, followed by returning the cultured product to a living body, and the method in which, during culturing of the bone marrow-derived cells, a differentiation-inducing factor is applied to the cells to thereby differentiate the cells to cells capable of forming a bone, and the differentiated cells are compensated in the missing portion of the bone.

However, the above method utilizing the bone marrow-derived cells (stem cells) suffers from the following problems. First, among the bone marrow-derived cells, it is not clear concerning what type of cells have a capability of differentiating to cells having a bone formation ability. Secondly, it is difficult to control the induction of the differentiation, and it is also difficult to determine the mutation conditions of the cells differentiated in vitro. Thirdly, at least two to four weeks are required in the growth of the bone marrow cells when the bone marrow cells are cultured under in vitro conditions. Fourthly, significant labor and large-sized equipment are required in the seeding and treating of the cells at a large scale since the method of adhering the bone marrow-derived cells to the scaffold material must be carried out under treatment circumstances which are completely separated from those of other patients. Since the implanting bone materials produced upon seeding of the bone marrow-derived cells and others as a starting material are all unique, it is difficult to provide different implanting bone materials having the same quality and specification.

In view of mass production of implanting bone materials, it becomes possible to produce the bone materials efficiently while ensuring good quality when the materials used as the bone materials are uniform. Namely, if it is expected to supply a large amount of implanting bone material, produced upon combining the cells and the implanting bone materials, it is desirable to develop the material having a bone formation capability which is comparable to that of the transplant bone, obtained from the living body, by using uniform cells. Formation of the material having such a bone formation capability can be effectively carried out by utilizing established cell lines. At present, some established cell lines such as MC3T3-E1 and the like are well-known in the art, but a practically usable implanting bone material has not yet been developed, even if the cells having the bone formation capability can be utilized.

### SUMMARY OF THE INVENTION

The present invention provides an implanting bone material having no toxicity which is safe in the body as well as having a high mechanical strength at the initial fixation, while simultaneously showing the properties of a living body. The present invention also provides a method of producing such an implanting bone material in a simplified method, in large amounts and with high efficiency.

According to an aspect of the present invention, a composite body of an inorganic compound and cells is provided, including animal cells filled with fine particles of the inorganic compound, and the fine particles of the inorganic compound adhered to an outer portion of the animal cells.

It is desirable for the fine particles of the inorganic compound to have an average particle size of 10 nm to 10 μm.

It is desirable for the fine particles of the inorganic compound to be coated with a polymeric nitrogen compound.

It is desirable for the polymeric nitrogen compound to be a number average molecular weight of 800 to 100,000.

It is desirable for the polymeric nitrogen compound to include a primary amino group, a secondary amino group and a tertiary amino group in a repeating structure unit thereof.

It is desirable for the polymeric nitrogen compound to be a straight chain, branched chain or cyclic polyamine compound or a mixture thereof.

It is desirable for the polyamine compound to be polyethylene imine.

It is desirable for the fine particles of the inorganic compound to include one of a calcium phosphate compound and metal oxide.

It is desirable for the calcium phosphate compound to be hydroxyapatite.

It is desirable for the metal oxide to be aluminum oxide, silica, magnesium oxide or ferric oxide.

In an embodiment, a method of producing a composite body of an inorganic compound and cells is provided, including cultivating animal cells in a culture medium containing fine particles of the inorganic compound, and separating a solid component from the cultivated product.

It is desirable for the inorganic compound to be contained in a concentration of 1.0 pg/cell to 1.0 ng/cell with respect to the animal cells.

It is desirable for the cultured animal cells to be deactivated after separation of the solid component from the cultivated product.

It is desirable for the deactivation treatment to include one of a heating treatment and a chemical treatment.

It is desirable for the chemical treatment includes an immobilization of a protein with an aldehyde compound or a dimaleimide compound.

It is desirable for the fine particles of the inorganic compound to be coated with a polymeric nitrogen compound.

It is desirable for the polymeric nitrogen compound to have a number average molecular weight of 800 to 100,000.

It is desirable for the polymeric nitrogen compound to include a primary amino group, a secondary amino group and a tertiary amino group in a repeating structure unit thereof.

It is desirable for the polymeric nitrogen compound to be one of a straight chain, a branched chain and cyclic polyamine compound, or a mixture thereof.

It is desirable for the polyamine compound to be polyethyleneimine.

It is desirable for the fine particles of the inorganic compound to include one of a calcium phosphate compound and metal oxide.

It is desirable for the calcium phosphate compound to be hydroxyapatite.

It is desirable for the metal oxide to be one of aluminum oxide, silica, magnesium oxide and ferric oxide.

According to the present invention, a large quantity of an implanting bone material can be provided which can exhibit properties similar to those of a living body material while having no toxicity, being safety in the living body, and also having good mechanical strength at the initial fixation of the bone material. Furthermore, according to the present invention, the implanting bone material having the above mentioned properties can be effectively produced in a large amount using the simplified method, without troublesome operations, by only utilizing the ingestion function of the cells which are inherent to the cells themselves, regardless of the presence or absence of the bone formation capability of the cells which are used as a starting substance.

As described above, the present invention was developed to provide a novel implanting bone material. However, the resulting composite body having an inorganic compound and cells can be applied to various applications such as a diagnostic agent and the like in which it is desired to ensure a good shape strength, while ensuring the properties of the living body material, in addition to the application as an implanting bone material.

The present disclosure relates to subject matter contained in Japanese Patent Application No. 2006-37554 (filed on Feb. 15, 2006) which is expressly incorporated herein in its entirety.

### BRIEF DESCRIPTION OF THE DRAWINGS

FIG. 1 is a transmission electron microscopic (TEM) photograph (×400) of the composite body of hydroxyapatite particles and cells, MDCK-HA, produced in Example 1;

FIG. 2 is a TEM photograph (×1,800) of MDCK-HA;

FIG. 3 is a scanning electron microscopic (SEM) photograph (×4,500) of MDCK-HA showing the HA particles as is attached to and is impregnated into the MDCK cells;

FIG. 4 is a SEM photograph (×3,500) of MDCK-HA showing the MDCK cells in the process of importing the HA particles;

FIG. 5 is a TEM photograph (×7,000) of the HA nanoparticles as a single body;

FIG. 6 is a TEM photograph (×400) of the MDCK cell as a single body;

FIG. 7 is a TEM photograph (×1,200) of the composite body (HOS-HA) produced in Example 2;

FIG. 8 is a TEM photograph (×1,500) of the dried product of the composite body (HOS-HA) produced in Example 2;

FIG. 9 is a TEM photograph (×3,000) of the composite body of silica particles and cells produced in Example 3;

FIG. 10 is a TEM photograph (×7,000) of the silica particles as a single body;

FIG. 11 is a TEM photograph (×700) of the composite body of alumina particles and cells produced in Example 4; and

FIG. 12 is a TEM photograph (×7,000) of the alumina particles as a single body.

### DESCRIPTION OF THE PREFERRED EMBODIMENTS

The present invention is further described with reference to the following embodiments. Note, however, that the present invention is not restricted to these embodiments.

The composite body of an inorganic compound and cells according to the present invention includes animal cells filled with fine particles of the inorganic compound, and the fine particles of inorganic compound adhered to an outer portion of the animal cells.

The animal cells used herein are not restricted to the specified cells so long as they can maintain their ingestion effect (function). The animal cells include, for example, canine kidney-originated cell strains (hereinafter abbreviated to MDCK), osteoblast sarcoma cell strains (hereinafter abbreviated to HOS), and others.

The fine particles of the inorganic compound include those of calcium phosphate compounds or metal oxides. Furthermore, the inorganic compound may be used alone or in combination. The calcium phosphate compounds which can be used herein are those having a Ca/P ratio of 1.0 to 2.0, and include, e.g., a variety of apatite compounds such as hydroxyapatite, fluorinated apatite and the like, calcium primary phosphate, calcium secondary phosphate, tricalcium phosphate, tetracalcium phosphate, and others. These apatite compounds may be used alone or in combination.

Furthermore, metal oxides include aluminum oxide, silicon dioxide, magnesium oxide, titanium oxide, ferric oxide (hematite), and others.

It is desirable for the fine particles of the inorganic compound to have an average particle size of about 10 nm to 10 μm. If the fine particles have an average particle size less than 10 nm, it is difficult to produce these fine particles at a mass-production level. On the other hand, if the fine particles have an average particle size above 10 μm, it is difficult to incorporate the fine particles into the animal cells utilizing the ingestion function of the cells.

Furthermore, the fine particles of the inorganic compound may be used without relying upon the conditions of the particles, e.g., the fine particles may have a state in which nanoparticles are adhered thereto (i.e., particles having a smaller particle size adhered to particles having a larger particle size) or a state in which nanoparticles are agglomerated to form the fine particles. Furthermore, the fine particles may be those produced using any conventional method.

One example of the fine particles of the inorganic compound according to the present invention will be now described with reference to the production of nanoparticles of the calcium phosphate compound which was developed by the inventor, and others, of this application. The nanoparticles of the calcium phosphate compound can be produced by thermally treating the calcium phosphate compound to produce particles of the calcium phosphate compound. The resulting particles are dispersed and crushed in an organic solvent to produce a dispersion of the calcium phosphate compound. The resulting dispersion is then centrifuged to obtain a supernatant thereof. The supernatant may be further dried, if necessary.

It is desirable that the calcium phosphate compound used in the above process is produced in accordance with any well-known method, and then dried prior to its thermal treatment, and, if necessary, the compound may be granulated. The granulation process can be carried out by using any well-known method. It is desirable that the compound is spray-dried to form a porous body having a volume ratio of not less than 70%.

Furthermore, in the above production method of the calcium phosphate compound, at the first stage thereof, the calcium phosphate compound is thermally treated using a well-known heating device to give a thermal history to the compound. The thermal treatment temperature is not restricted, but it is desirable that the thermal treatment is carried out at a temperature of about 400 to 1050° C. A lower temperature than 400° C. will result in poor strength of the particles, thus lowering their usable strength. On the other hand, a temperature higher than 1050° C. will result in partial or complete sintering of the particles, thereby lowering the yield of the nanoparticles.

After the thermal treatment, the resulting particles are dispersed in an organic solvent by subjecting the particles to the crushing treatment including, for example, ultrasonic treatment, homogenizer, shaking machine, mortar and the like. It is desirable for the organic solvent to be a polar organic solvent, and for the polar organic solvent to includes, for example, alcohols such as ethanol and isopropanol, ethers such as 2-ethoxyethanol, acetonitril, tetrahydrofuran, dimethylsulfoxide, and others.

Before the crushing treatment process, the particles of the calcium phosphate compound may be treated in a ball mill. The ball mill is generally operated with balls (media) for grinding the sample particles through the rubbing motion thereof; however, if the ball mill is operated without using balls, it is possible to produce nanoparticles with a spherical body having an aspect ratio approaching 1 at a high yield. In this milling process, it is desirable that milling is carried out in the absence of an organic solvent, and the like (under the conditions that only the particles of the calcium phosphate compound are contained in a milling pot), i.e., under the dried conditions (hereinafter, this process is referred to as a dry milling process).

The resulting dispersion of the calcium phosphate compound is then subjected to a centrifugal treatment process. As a result, the dispersion is fractionated into a supernatant which has the nanoparticles of the calcium phosphate compound dispersed in a phase of the organic solvent, and precipitates of the calcium phosphate compound which have a larger particle size than that of the nanoparticles. Thereafter, the organic solvent is evaporated from the supernatant to obtain the nanoparticles of the calcium phosphate compound.

It is desirable for the fine particles of the inorganic compound are coated with a polymeric nitrogen compound. Specifically, it is desirable that the fine particles of the inorganic compound are coated with a polymeric nitrogen compound through the solid acid point (electron vs. acceptor) of the inorganic compound to which at least one amino group of the nitrogen compound is bonded.

The fine particles of the inorganic compound coated with the polymeric nitrogen compound are produced by mixing the fine particles of the inorganic compound and the polymeric nitrogen compound in a solution. The resulting fine particles of the inorganic compound coated with the polymeric nitrogen compound is centrifuged to produce a supernatant which is then dried to obtain the fine particles of the inorganic compound coated with the polymeric nitrogen compound. Since an improved dispersion property is obtained in the resulting fine particles along with maintenance of their small particle size, the fine particles can be more easily incorporated into animal cells in comparison with the fine particles of the inorganic compound having no coating.

Polymeric nitrogen compounds having at least two amino groups, selected from a primary amino group, a secondary amino group and a tertiary amino group, in a molecular thereof can be used as the polymeric nitrogen compound, for example, a polyamine compound can be used. The polyamine compound may be any one of straight chain polyamines, branched chain polyamines, cyclic polyamine compounds or a mixture thereof. It is desirable that a large amount of primary, secondary and tertiary amino groups are contained in a repeating unit of the molecule, because the amino groups can provide an increased number of the bonding sites to the fine particles of the inorganic compound.

The polyamine compounds described above include, for example, polyethylene imine, polylysine and others. Among these polyamine compounds, polyethylene imine is the desirable. Polyethylene imine is a polymer of ethylene imine, a molecular structure of which can be varied depending upon the amino group capable of concerning the polymerization reaction.

It is desirable that a number average molecular weight of the polyamine compounds (polymeric nitrogen compounds) is in a range of about 800 to 100,000. When the number average molecular weight is less than 800, it becomes difficult to effectively coat the fine particles of the inorganic compound with the polyamine compound. As a result, the fine particles of the inorganic compound cannot be effectively incorporated into the animal cells. On the other hand, when the number average molecular weight is above 100,000, large aggregated blocks are produced as the result of bonding of the adjacent polyamine compounds. As a result, it becomes difficult to incorporate the fine particles of the inorganic compound into the animal cells.

Moreover, the concentration of the solution containing the polymeric nitrogen compound cannot be simply determined because of variation depending upon the type, amount and surface area of the fine particles of the inorganic compound to be coated, and other factors. However, an excessively reduced concentration of the polymeric nitrogen compound has a tendency to bond to the fine particles of the inorganic compound, but they are insufficient to fully coat the particles, thereby causing an aggregation between the adjacent particles which results in insufficient incorporation of the particles into the animal cells. On the other hand, the excessively increased concentration of the polymeric nitrogen compound has a tendency of bonding to each other, in addition to the bonding of the nitrogen compound to the fine particles of the inorganic material, thereby forming a micellar structure which causes difficulties in the incorporation of the particles in the animal cells. Accordingly, it is desirable that the concentration of the solution of the polymeric nitrogen compound is suitably determined depending upon the type, amount and surface area of the fine particles of the inorganic compound to be coated and other factors.

The production of the composite body consisting of the organic compound and the cells is described hereinafter.

The animal cells are previously subjected to the cell culturing process to proliferate the cells to a cellular mass having a desired number of cells. Thereafter, a cellular mass of the animal cells is cultivated in a culture medium containing fine particles of the inorganic compound. The cultivation process can be carried out under conditions which are conventional in the cultivation of the animal cells. It is desirable that the fine particles of the inorganic compound are used in a concentration of about 1.0 pg/cell to 1.0 ng/cell for the animal cells.

After the cultivation process, the solid content is separated from the culture medium to obtain a composite body of the inorganic compound and the cells. Note that, after separation of the solid component, if the cells of the composite body are compatible with the living body, the composite body may be used as the living cells. Alternatively, the animal cells may be subjected to deactivation treatment if desired.

The deactivation treatment is intended to stop the biogenic action of the animal cells and the functions of proteins and others in the cells, and includes a heating treatment, a chemical treatment and other similar treatments. The chemical treatment includes an immobilization of a protein with an aldehyde compound or a dimaleimide compound.

Furthermore, the resulting composite body may be heated to obtain a shaped body. Moreover, the shaped body may be further dried to obtain a strengthened shaped body.

### EXAMPLES

The present invention will be further described with reference to the examples thereof. Note, however, that the present invention is not restricted to these examples.

The composite body of an inorganic compound and cells produced in the following examples was evaluated in accordance with the following method.

### (A) Observation of the Composite Body

The composite body of an inorganic compound and cells according to the invention was observed by using a transmission electron microscope (TEM). The transmission electron microscope used herein is H-7600 (trade name) produced by Hitachi Co., Ltd. Note that before the TEM observation, the composite was immobilized and embedded with glutaraldehyde.

### (B) Determination of the Constituent Atoms

The composite body of an inorganic compound and cells was dry-treated at 100° C., and the constituent atoms thereof were analyzed (determined) by using an energy dispersion X-ray electron micrograph (EDX). The EDX used herein is S-4300 (trade name) produced by Hitachi Co., Ltd.

### Example 1

The method for producing a composite body of an inorganic compound and cells used in this example is described hereinafter.

MDCK (canine kidney-originated cell strains) cells were used as the animal cells. A 4 ml of culturing solution containing cultured MDCK cells and 3 mg/ml of fine particles of calciumphosphate was added to a flask of the volume of 225 cm², and the culturing solution was further added to control the volume to 60 ml. The culturing solution was then cultured under conditions of 37° C. and 5% CO₂ for 29 hours. Note that the culturing solution used herein is a mixture of the MEM culture medium and FBS (10 vol. %).

Calcium phosphate used herein is hydroxyapatite (hereinafter, referred to as HA) produced by the following method. Namely, a slurry containing hydroxyapatite produced in a wet process was dried at 200° C. in a spray dry apparatus, and granulated. Thereafter, the granulated HA product was classified to obtain the HA particles having an average particle size of 10 μm. The resulting HA particles were added to an electric furnace, and their temperature was increased at a rate of 50° C./h to 850° C. which was maintained for 4 hours. HA particles were thus obtained.

A 1.0 g of the resulting HA particles were added to a pot made of zirconia having an inner volume of 45 ml, and was dry-milled at a rotation speed of 800 rps for 3 hours in a planetary ball mill (Fritsch Co., Ltd.; P-7). Note that the dry-mill treatment was carried out in the planetary ball mill without using the medium (balls) in a pot thereof. After the dry-mill treatment, the HA particles were removed from the mill to obtain the dry-milled HA particles.

A 30 ml of the solution containing 10 mg/ml of polyethylene imine (hereinafter, referred to as PEI) was added to the HA particles to prepare a dispersion of the HA particles. The resulting dispersion was subjected to an ultrasonic treatment process (output: 180 W; 5 minutes) in an ultrasonic generation apparatus (produced by TAITEC Co.; VP-30S). The HA particles were crushed and the surface of the HA particles were coated with PEI. Thereafter, isopropanol was added to make a total amount of 10 ml. The product was centrifuged at 4100×g for 10 minutes. After the centrifugal treatment, the supernatant, i.e., dispersion of the PEI-coated HA nanoparticles was obtained.

An average particle size of the HA nanoparticles was about 125 nm. The determination of the average particle size was made according to the dynamic scattering method using a submicron particles analyzer N5 (produced by Beckman Coulter Co., Ltd.).

After the culturing process, a surface of the MDCK cells was washed off with a phosphate buffer solution to remove the HA nanoparticles not adsorbed on the MDCK cells. The resulting composite body (hereinafter, referred to as MDCK-HA) of HA nanoparticles and MDCK cells was removed with a scraper from the flask, and then centrifuged at 5,000 rpm for 5 minutes. The solid content precipitated upon centrifugation was collected as a composite body of the HA nanoparticles and the cells.

The composite body of the inorganic compound and the cells in this example was evaluated in accordance with the following method.

The composite body of the inorganic compound and the cells was immobilized with 2% glutaraldehyde (hereinafter, GA), and centrifuged. The immobilized composite body was washed with distilled water, and dried at 100° C. Thereafter, the dried product was heated to 1,300° C. in a thermal analysis apparatus (produced by Rigaku Co.; TGS120). Heating was stopped when the weight of the product was maintained at a constant level, followed by cooling. The weight of the product before and after heating was measured.

After heating in the thermal analysis apparatus, the constituent atoms of the composite body of the inorganic atom and the cells were determined by using an energy dispersion X-ray electron micrograph (hereinafter, referred to as EDX; produced by Hitachi; S-4300).

The results for different analysis processes of the composite body of the inorganic compound and the cells in this example are summarized as follows.

### (A) Observation of the Composite Body

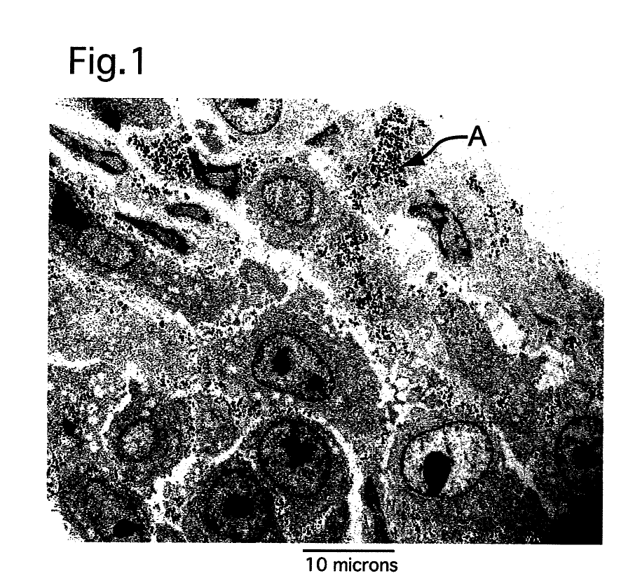

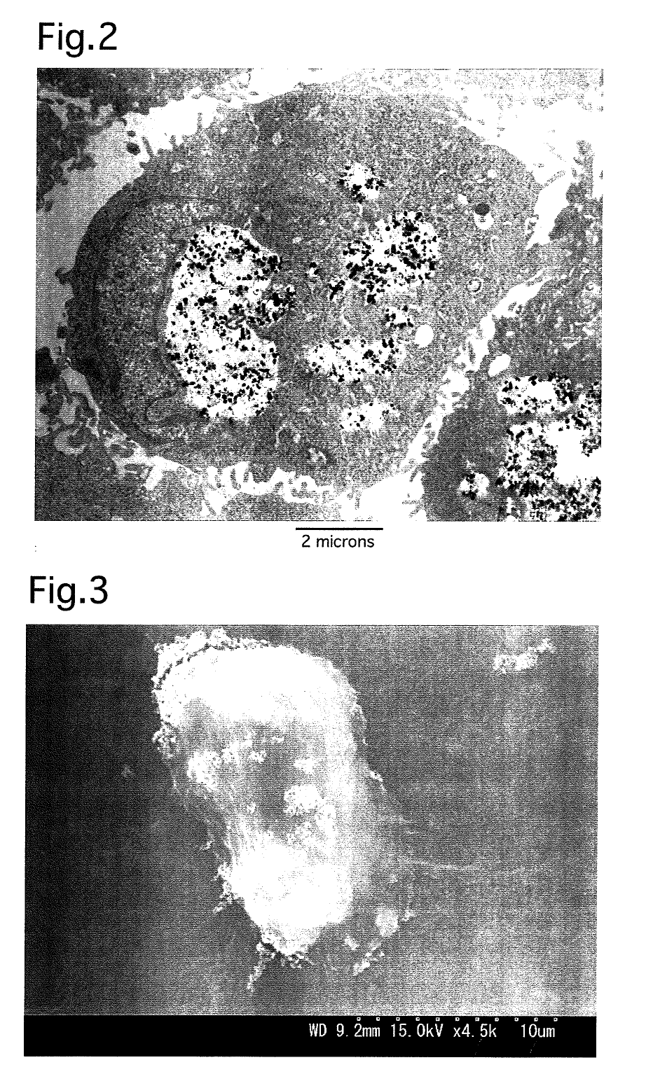

FIG. 1 is a transmission electron microscope (TEM; magnification: ×400) photograph of MDCK-HA in this example. The nanoparticles of hydroxyapatite (for example, black points A in FIG. 1) were observed in an inner and outer portion of the MDCK cells. Namely, it was confirmed that the HA nanoparticles were sufficiently incorporated in the cells. FIG. 2 is a TEM photograph of MDCK-HA having a magnification of ×1,800 which clearly shows the HA particles.

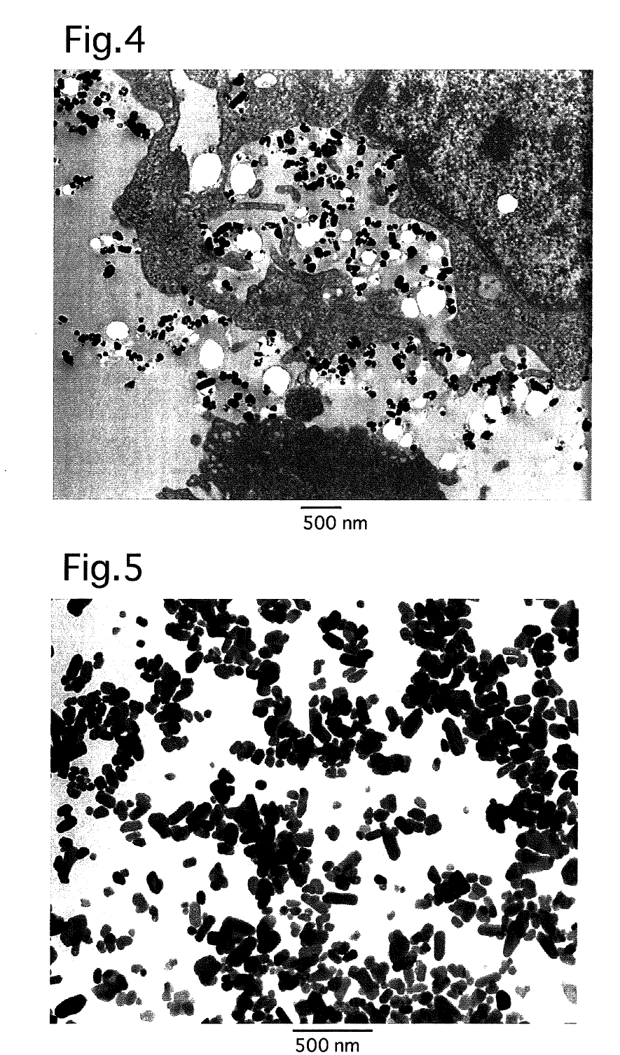

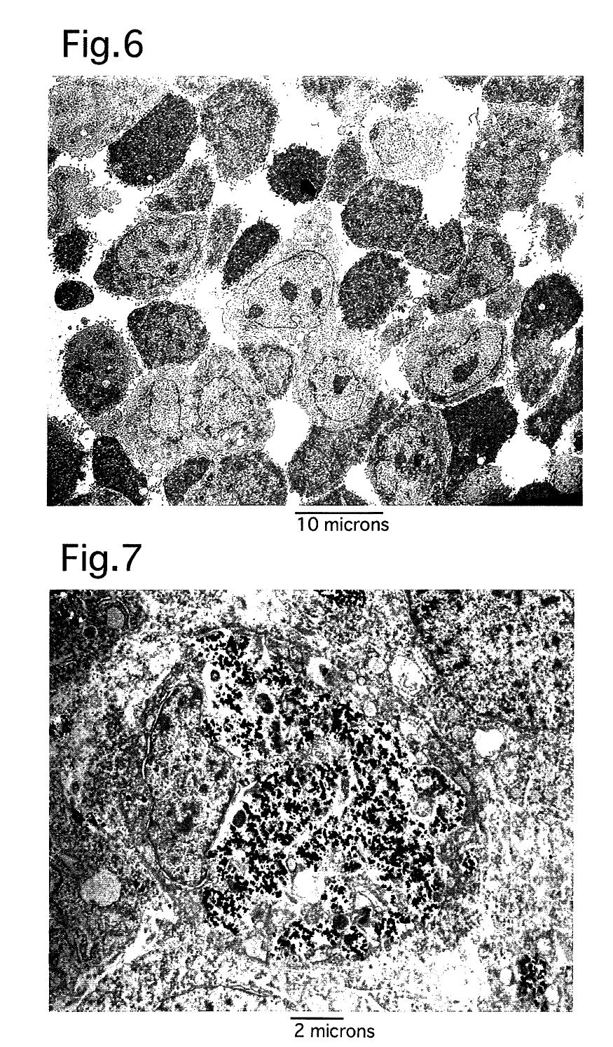

FIG. 3 is a SEM photograph (magnification: ×4,500) of MDCK-HA, from which it is appreciated that the HA particles are in the process of being adhered to and incorporated into the MDCK cells. FIG. 4 is a SEM photograph (magnification: ×3,500) of MDCK-HA showing that the MDCK cells are in the process of incorporating the HA particles therein. Furthermore, FIG. 5 is a TEM photograph (magnification: ×7,000) of the HA nanoparticles as a single body, and FIG. 6 is a TEM photograph (magnification: ×400) of the MDCK cell as a single body;

### (B) Variation of the Weight

Table 1 shows a variation of the weight in the composite body (MDCK-HA) of the HA nanoparticles and the cells before and after heating.

TABLE 1

| | Before | After | |
|---|---|---|---|
| | heating (A; | heating (B; | Difference |
| | mg) | mg) | (A − B; mg) |
| MDCK | 18.32 | 0.30 | 18.02 |
| MDCK-HA | 27.32 | 6.40 | 20.92 |

Since the loss in the weight of the HA nanoparticles is not caused by heating, the weight of HA can be determined depending upon only the weight of the MDCK cells. Upon subtracting the weight of HA from that of MDCK-HA, the weight of MDCK before heating was determined to be 21.22 mg. Furthermore, the weight of HA was determined by calculating a weight of the heated MDCK cells from the difference in the weight of the single body of the MDCK cells before and after heating.

TABLE 2

| | MDCK | MDCK | |
|---|---|---|---|
| | Before heating | After heating | HA weight |
| | (mg) | (mg) | (mg) |
| MDCK | 18.32 | 0.30 | 0.00 |
| MDCK-HA | 21.22* | 0.35** | 6.05 |

Note:
*= 27.32 − 6.40 + 0.30
**= 21.22 × 0.30/18.32

### (C) Determination of the Constituent Atoms

Table 3 shows the results of the analysis made by EDX for MDCK-HA (magnification of the determination: ×1,200).

TABLE 3

| | | Atomic no. conc. |
|---|---|---|
| Element | Weight conc. (%) | (%) |
| O | 38.09 | 60.52 |
| P | 16.53 | 13.57 |
| Ca | 30.10 | 19.09 |
| Others | 15.28 | 6.82 |

Furthermore, Table 4 is a comparative example showing the results of the analysis made by EDX for the single body of the HA nanoparticles.

TABLE 4

| | | Atomic no. conc. |
|---|---|---|
| Element | Weight conc. (%) | (%) |
| O | 37.36 | 58.02 |
| P | 20.47 | 16.25 |
| Ca | 41.40 | 25.40 |
| Others | 0.37 | 0.33 |

In Table 3, it is considered that the column “others” of MDCK-HA includes the residues generated during the production of MDCK-HA, a trace amount of carbons or the remainder of the culturing solution.

It is appreciated from Tables 3 and 4 that the composition and the concentration are substantially equivalent and the HA nanoparticles are contained in MDCK-HA. Furthermore, it is deemed that the HA nanoparticles have been incorporated in the MDCK cells, because after the culturing process a surface of MDCK-HA was washed to remove the unincorporated HA nanoparticles from MDCK-HA.

### Example 2

In this example, osteoblast sarcoma cell strains (hereinafter, referred to as HOS) were used instead of MDCK of Example 1. The HOS cells were cultured under the conditions similar to those of Example 1 to obtain the desired number of the cells. A 2.25 ml culturing solution containing 18 mg/ml of the HA nanoparticles (average particle size: about 125 nm) in the cultured HOS cells was added to a flask having a volume of 225 cm², and the content was cultured under conditions of 37° C. and 5% CO₂ for one hour. Thereafter, the surface of the HOS was washed off with a phosphate buffer solution, a fresh culturing solution was further added to the cultured content, and the content was further cultured for 20 hours. After the culturing process, trypsin was added and the product was separated from the flask to obtain a composite body of HA nanoparticles and HOS cells (hereinafter, referred to as HOS-HA).

Note that the culturing solution used in the above process is a mixture of FBS (10 Vol. %) and NEAA (1 Vol. %) in the MEM culture medium.

In the evaluation of the composite body of the HA nanoparticles and the HOS cells produced in this example, the resulting composite body was centrifuged at 5,000 rpm for 5 minutes for pelleting down the composite body. The composite body was immobilized with 2% glutaraldehyde (hereinafter, GA), embedded, sliced and observed with a TEM (FIG. 7).

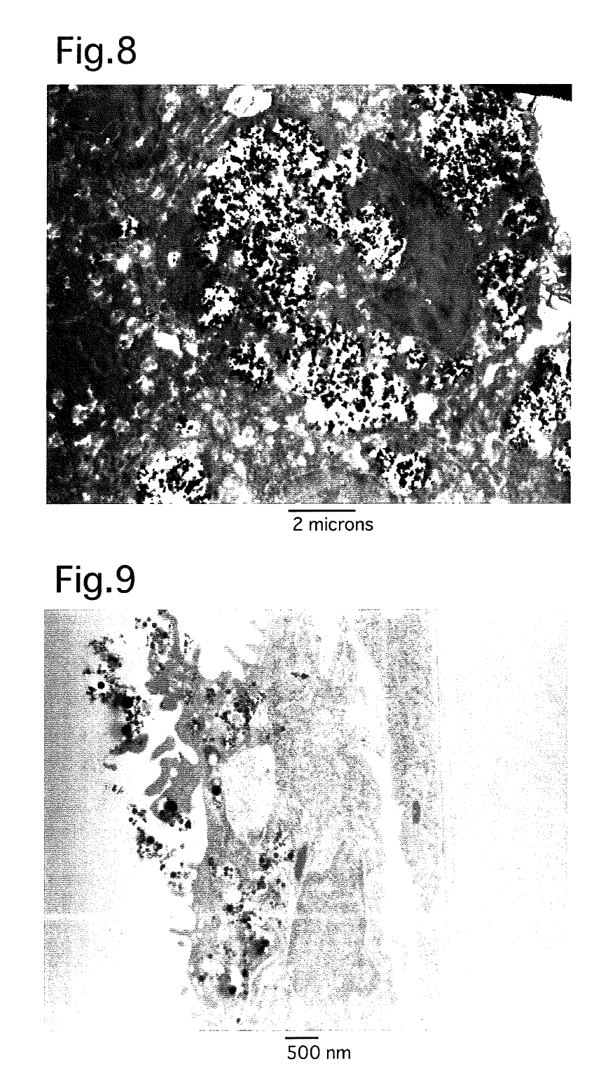

Furthermore, after the composite body was centrifuged at 5,000 rpm for 5 minutes for pelleting down the composite body, the composite body was immobilized with GA, dried at 100° C. in an oven for one night, embedded, sliced and observed with a TEM (FIG. 8).

The results for different analysis processes of the composite body of the inorganic compound and the cells in this example are summarized as follows.

### (A) Observation of the Composite Body

FIG. 7 shows a TEM photograph (×1,200) of HOS-HA. The HA nanoparticles (for example, black points B in FIG. 7) were observed in an inner and outer portion of the MDCK cells as being incorporated in the cells. FIG. 8 is a TEM photograph (×1,500) of HOS-HA which was obtained by immobilizing with GA and drying at 100° C. after the HOS-HA was pelleted down via centrifugation.

It is clearly observed from these drawings that the inorganic compound was incorporated in the cells.

The composite body shown in FIG. 7 is considered to be effective since the cells-biocompatible particles can act as one living cell.

The composite body shown in FIG. 8 demonstrates that when it is not alive, i.e., for example, when it is used after drying, the composite body can be used as cells having particles incorporated therein.

When the composite body of the cells having incorporated therein the HA particles is used as a biomaterial such as an implanting bone material as described above, in the case where the composite body results in problems when being used in treatments such as allotransplantation which is generally considered to have poor biocompatibility, the composite can be utilized after application of drying and other treatments. Alternatively, the composite body may be modified upon heating thereof to a biocompatible material which can be stocked.

### Example 3

In this example, the silica particles (SiO₂; produced by C.I. Chemical Co.; Nanotek Powder) were used instead of the HA nanoparticles of Example 1. The average particle size of the silica particles was 100 nm.

A culture medium containing 100 g/ml of the silica particles was added to the MDCK cells cultured for one night on a multi-well plate (produced by Becton Dickinson Co.) for culturing 12 well cells, and was cultured under the conditions of 37° C. and 5% CO₂ for 2 hours. After the culturing process, the culture medium was sucked and removed. The composite body was immobilized with 2% GA, embedded, sliced and observed with a TEM.

The results for different analysis processes of the composite body of the silica and the cells in this example are summarized as follows.

FIG. 9 is a TEM photograph (×3,000) of the composite body produced in this example. It was observed that the silica particles (for example, black points C in FIG. 9) were incorporated in an inner and outer portion of the MDCK cells.

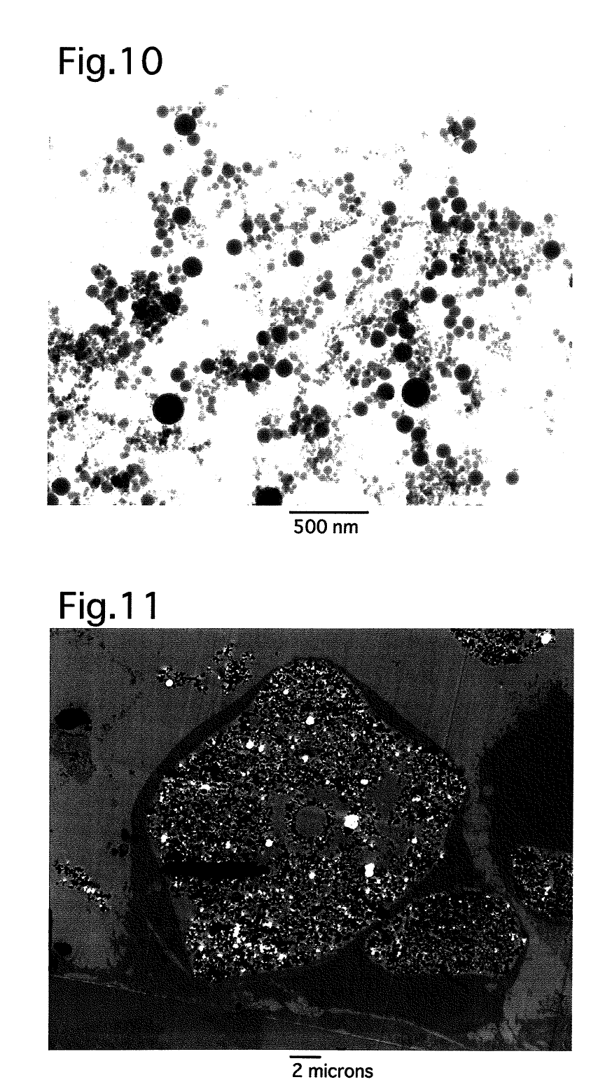

For the purpose of comparison, FIG. 10 shows a TEM photograph (×7,000) of the silica particles as a single body.

### Example 4

In this example, the alumina particles (Al₂O₃; produced by C.I. Chemical Co.; Nanotek Powder) were used instead of the HA nanoparticles of Example 1. The average particle size of the silica particles was 80 nm.

As in Example 3, a culture medium containing 2.3 mg/ml of the alumina particles was added to the MDCK cells cultured for one night on a multi-well plate (produced by Becton Dickinson Co.) for culturing 12 well cells, and was cultured under the conditions of 37° C. and 5% CO₂ for 20 hours. The cultured product was immobilized with 2% GA, embedded, sliced and observed with a TEM.

FIG. 11 is a TEM photograph (×700) of the composite body produced in this example. It was observed that the alumina particles (for example, black points D in FIG. 11) were incorporated in an inner and outer portion of the MDCK cells.

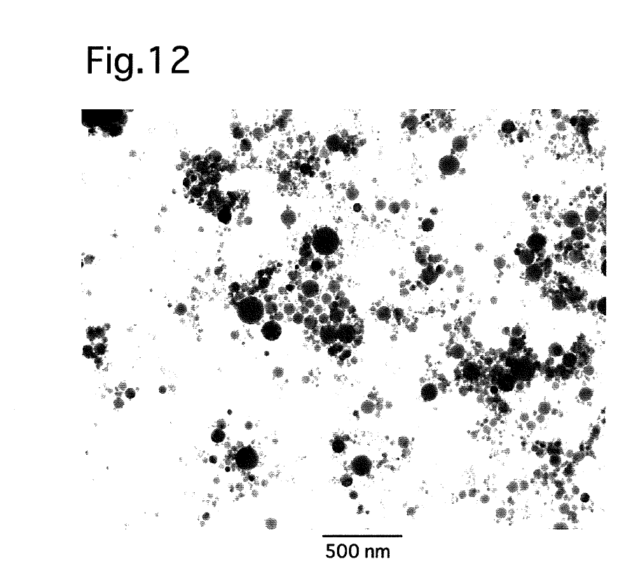

For the purpose of comparison, FIG. 12 shows a TEM photograph (×7,000) of the alumina particles as a single body.

Although the invention has been described with reference to particular means, materials and embodiments, it is to be understood that the invention is not limited to the particulars disclosed and extends to all equivalents within the scope of the claims.

What is claimed is:

1. A composite body of an inorganic compound and cells comprising:
animal cells filled with fine particles of said inorganic compound; and
said fine particles of said inorganic compound adhered to an outer portion of the animal cells.

2. The composite body according to claim 1, wherein said fine particles of said inorganic compound have an average particle size of 10 nm to 10 μm.

3. The composite body according to claim 1, wherein said fine particles of said inorganic compound are coated with a polymeric nitrogen compound.

4. The composite body according to claim 3, wherein said polymeric nitrogen compound has a number average molecular weight of 800 to 100,000.

5. The composite body according to claim 3, wherein said polymeric nitrogen compound comprises a primary amino group, a secondary amino group and a tertiary amino group in a repeating structure unit thereof.

6. The composite body according to claim 3, wherein said polymeric nitrogen compound is a straight chain, branched chain or cyclic polyamine compound or a mixture thereof.

7. The composite body according to claim 6, wherein said polyamine compound is polyethylene imine.

8. The composite body according to claim 1, wherein said fine particles of said inorganic compound comprises one of a calcium phosphate compound and metal oxide.

9. The composite body according to claim 8, wherein said calcium phosphate compound comprises hydroxyapatite.

10. The composite body according to claim 8, wherein said metal oxide comprises aluminum oxide, silica, magnesium oxide or ferric oxide.

11. A method of producing a composite body of an inorganic compound and cells comprising:
cultivating animal cells in a culture medium containing fine particles of the inorganic compound; and
separating a solid component from the cultivated product.

12. The production method according to claim 11, wherein said inorganic compound is contained in a concentration of 1.0 pg/cell to 1.0 ng/cell with respect to the animal cells.

13. The production method according to claim 11, wherein said cultured animal cells are deactivated after separation of said solid component from said cultivated product.

14. The production method according to claim 13, wherein said deactivation treatment comprises one of a heating treatment and a chemical treatment.

15. The production method according to claim 14, wherein said chemical treatment comprises an immobilization of a protein with an aldehyde compound or a dimaleimide compound.

16. The production method according to claim 11, wherein said fine particles of the inorganic compound are coated with a polymeric nitrogen compound.

17. The production method according to claim 16, wherein said polymeric nitrogen compound has a number average molecular weight of 800 to 100,000.

18. The production method according to claim 16, wherein said polymeric nitrogen compound comprises a primary amino group, a secondary amino group and a tertiary amino group in a repeating structure unit thereof.

19. The production method according to claim 16, wherein said polymeric nitrogen compound comprises one of a straight chain, a branched chain and cyclic polyamine compound, or a mixture thereof.

20. The production method according to claim 19, wherein said polyamine compound comprises polyethyleneimine.

21. The production method according to claim 11, wherein said fine particles of said inorganic compound comprise one of a calcium phosphate compound and metal oxide.

22. The production method according to claim 21, wherein said calcium phosphate compound comprises hydroxyapatite.

23. The production method according to claim 21, wherein said metal oxide comprises one of aluminum oxide, silica, magnesium oxide and ferric oxide.
